(19) United States
(12) Patent Application Publication
(10) Pub. No.: US 2018/0311417 A1
(43) Pub. Date: Nov. 1, 2018

(54) PHARYNGEAL-ENTERIC TUBE COMBINATION

(71) Applicant: Cedars-Sinai Medical Center, Los Angeles, CA (US)

(72) Inventor: Axel ROSENGART, Los Angeles, CA (US)

(73) Assignee: Cedars-Sinai Medical Center, Los Angeles, CA (US)

(21) Appl. No.: 15/772,673

(22) PCT Filed: Nov. 4, 2016
(86) PCT No.: PCT/US16/60630

§ 371 Date: May 1, 2018

Related U.S. Application Data
(60) Provisional application No. 62/251,490, filed on Nov. 5, 2015.

(51) Int. Cl. A61M 1/00; A61J 15/00; A61M 16/10

(52) U.S. Cl. CPC A61M 1/0084; A61M 1/0086; A61J 15/0073; A61M 1/0023; A61M 16/1005; A61M 2205/3303; A61M 2210/1046; A61M 2207/00; A61M 2210/1028; A61M 2202/0208; A61M 2210/1053; A61J 15/003

(57) ABSTRACT

The disclosed device, method, and system provide a simple, safe, effective and widely applicable method-device to suction the throat in patients with absence or incompetent swallowing reflexes. For instance, disclosed is a combination enteric feeding and pharyngeal suctioning tube that both pumps food to a patient's stomach while suctioning a patient's throat to remove pharyngeal secretions. The suctioning tube may be an attachment to a feeding tube that terminates just at or above the esophageal opening when connected to the feeding tube. The end of the pharyngeal suctioning tube may include perforations that allow the pharyngeal secretions to be vacuumed into the suctioning portion for removal.

### FIELD OF THE DISCLOSURE

The present disclosure is directed to a device, a method, and a system for suctioning of the pharynx and enteric access for feeding and suctioning of the gastrointestinal tract and supply of oxygen to the upper airways and lungs. More particularly, the present disclosure relates to a device, a method, and a system for pumping food to a patient's stomach while suctioning a patient's throat to remove pharyngeal secretions.

### BACKGROUND OF THE DISCLOSURE

The following description includes information that may be useful in understanding the present disclosure. It is not an admission that any of the information provided herein is prior art or relevant to the presently claimed disclosure, or that any publication specifically or implicitly referenced is prior art.

Patients with a wide variety of illnesses (i.e., after a brain bleed, surgery, cancer, and the like) as well as individuals with chronic disorders of speech or swallowing motoric (i.e., after stroke) are at markedly increased risk to have oral secretions or orally ingested fluid inadvertently entering the trachea and the lungs, a process called respiratory aspiration of fluids (or simply, aspiration), rather than draining it into the digestive track (esophagus). Additionally, aspiration in these patients may also occur from stomach fluid inadvertently regurgitated into the throat (i.e., lying flat at nighttime, during coughing, or in reflux illness). Furthermore, acutely or chronically ill patients may be in demand of supplemental oxygen to improve breathing and increase oxygen content within the blood.

Aspiration events are overall extremely common; and they vary in the fluid amounts that enter the lungs (micro- to several milliliters). They cause pain, discomfort, vocal cord irritation, airway and lung infections and shortness of breath and are among the most common causes for lungs complications, leading to increased care costs and length of hospital stay. Aspiration events directly cause increased morbidity and mortality, lead to irritations of the sensitive, softer lower lung tissue areas (alveoli) with risk for chronic lung fibrosis and are difficult to detect and diagnose (especially recurrent, low fluid volume aspirations, so called micro-aspirations) before the aspiration becomes clinically evident (i.e., by florid infection or gas exchange problems). Furthermore, the demand for supplemental oxygen is also extremely common as many patients with various lung and non-lung illnesses suffer from low oxygen in the blood (hypoxemia). Hypoxemia, if severe, is treated with inserting a breathing tube into the windpipe (endotracheal intubation follow by mechanical ventilation); however, the most common forms are mild to moderate hypoxemia which are routinely treated with supplemental oxygen provided via nasal prongs or face mask. Unfortunately, oxygen supplementation provided via nasal prongs or face mask often do not sufficiently elevate the oxygen content inside the lungs as many patients breath through the mouth (hence, little use for nasal prongs) or have an incomplete seal with the face mask either due to the shape of the face or dislodgement from the nose/mouth openings because of discomfort.

Intermittent oral or, less often, nasal suctioning, performed via insertion of a suction catheter through the mouth or nose, is currently the only existing solution and means to decrease pharyngeal secretions. Alternatively, some medications are capable of drying out mucous membranes and decreasing saliva production; however, generally those are avoided due to frequent adverse effects, unpleasantness to the patient and modest efficacies. Patients in need for supplemental oxygen are treated with nasal prongs or face masks providing oxygen-enriched air to the upper airways and ultimately to the lung tissue while the patient is spontaneously breathing. Again, the actual amount of oxygen delivered to the lungs with these devices is much lower than applied due to by-mixture of room air and difficulties positioning the devices on the face. In addition, orally or nasally supplemented oxygen also dries out mucous membranes of the mouth and nose, respectively, which is unpleasant to the patient and increases the risks for infections.

### SUMMARY OF THE DISCLOSURE

However, intermittent oral suctioning: (1) requires, in the majority of patients, a vigilant caregiver day and night; (2) produces uncomfortable arousal each time a catheter is inserted into the throat; (3) elicits very frequently cough, gag, swallowing and apneic (breath holding) reflexes which are not only unpleasant experiences but also unwanted in patients with acute brain injury or healing wounds post-surgery, among many others; (4) can lead to bleeding and laceration of mucous membranes; and (5) requires a new, clean suction device replacement set at least every 24 hours resulting in ongoing daily material costs.

With respect to the need of supplemental oxygen the use of nasal prongs and face masks has certain limitations. Nasal prongs (1) often dislodge from the nostrils which is commonly neither noted by the patient nor the care taker exposing the patient to unwitnessed hypoxemic episodes; (2) are frequently irritating as they reside and jiggle within the highly sensitive mucosa of the nostril cavity; and (3) do not reliably provide supplemental oxygen as many patients are dominant mouth-breathers, especially during sleep. Face masks and tends are alternatives for supplying supplemental oxygen; however, they (1) are much more cumbersome to the patients as they are strapped over the face (via elastic cords around the head) to cover the mouth and nose and many patients report feelings of suffocation; (2) must be taken off for oral care and eating exposing the patient to hypoxemia; and (3) also dislodge frequently to slide sideward off the face or above the eyebrows.

Therefore, a method aiming at reducing the frequency and severity of aspiration events, increasing the comfort of oropharyngeal secretion removal, and to on-demand supply supplemental oxygen directly to the pharyngeal cavity without the need for facial oxygen delivery devices is of great clinical importance. The disclosed device, system, and methods aim at reducing the amount, rate and frequency of aspirations using a practical, minimally invasive approach by conveniently combining a new suction catheter with an already clinically commonly used feeding tube. It also aims at a convenient, safe and reliable method to provide supplemental oxygen directly to the patient's larynx (the entrance of the windpipe) if desired.

### Overview

The disclosed systems and methods provide a simple, safe, effective and widely applicable method-device to suction the throat in patients with absence or incompetent swallowing reflexes. Below is an anatomical diagram of the pharynx anatomy for reference:

Additionally, the disclosed systems and methods may provide supplemental oxygen directly the entrance of the larynx. For instance, disclosed is a combination enteric feeding and pharyngeal suctioning tube that both pumps food into a patient's stomach while suctioning a patient's throat to remove pharyngeal secretions. In some embodiments, the combination will also provide the ability to suction both the patient's throat and stomach simultaneously. The suctioning tube may be an attachment to a feeding tube that terminates just at or above the esophageal opening when connected to the feeding tube. The pharyngeal suctioning tube segment reaching from the back of the nose to the entry of the esophagus may include perforations that allow the pharyngeal secretions to be vacuumed into the suctioning portion for removal. The perforations in the pharyngeal tube may run longitudinally and in a sloped (twisted) pattern to avoid suction induced adherence to the mucosal wall of the throat.

In some embodiments, to administer supplemental oxygen, the pharyngeal tube can also be used to deliver supplemental oxygen directly into the opening of the larynx, the airway entry site. The pharyngeal tube segment reaching from the back of the nose to the entry of the larynx may include perforations that allow oxygen to be dispensed directly in front of the laryngeal entrance allowing oxygen to readily mix with the inspiratory air. In other embodiments, a third tube with a length similar to the pharyngeal tube may be utilized for delivering supplemental oxygen to the opening of the larynx while simultaneously suctioning of secretions can be continued.

The tubes may include coaxially arranged lumens with the pharyngeal suctioning lumen positioned as the outer lumen or external to the feeding tube lumen (i.e., “tube-in-tube” design). The pharyngeal lumen provides a path for secretions that include been vacuumed into the perforations to flow towards an external reservoir for disposal. The pharyngeal tube terminates at its distal end in a smooth, conical fashion, tapering into the feeding tube. The pharyngeal tube may include openings for suctioning and/or oxygen delivery that are within the length of the tube segment that reaches from the back of the nose to its conical ending and merging with the enteric feeding tube.

An enteric tube is usually inserted through the nose into the nasal passages, crossing the back of the throat (pharynx), entering through the food pipe (esophagus) to terminate in the stomach or further downstream within the intestines. The proposed devices and methods may include a second tube for pharyngeal suctioning fitted as an outer tube over the enteric tube while leaving a space for fluid flow between the outer circumference of the inner feeding tube and the inner circumference of the outer pharyngeal tube. In other embodiments, the suctioning tube may be adjacent to and non-coaxial with the enteric tube in a side-by-side relationship.

In other embodiments, a third tube which may include a similar (or identical) design as the pharyngeal suction tube may provide access to the back of the throat for supplemental oxygen delivery. For example, a three-piece combination tube may include the following components: (1) an inner tube for enteric feeding, (2) and outer tube that separated in 2 halves. The outer tube could include: (1) one half for suctioning which ends ½ to 1 inch below (or at same level) as the other half, and (2) the other half for supplying oxygen. The oxygen would be supplied continuously while suctioning would generally only be applied intermittently and on-demand. In some embodiments, feeding could be administered independent of providing oxygen and suctioning.

In still other embodiments, the enteric tube may include a larger diameter aspiration tube to, in some embodiments, quickly suction out stomach content, i.e., from a trauma victim to reduce risks of fluid regurgitation and aspiration, and the pharyngeal tube may only or primarily deliver high-flow oxygen to the larynx. This embodiment may be useful in emergency settings for trauma victims or other medical urgencies, as high flow pre-oxygenation can be extremely useful to keep blood oxygenation adequate in case the patient experiences hypoxemia or even breathing arrest (apnea).

In some embodiments, this tube-in-tube design may only include a short segment of the feeding/aspiration tube: for example, it may start at the nostril and conically end where the feeding tube enters the esophagus, for instance, a median length of 19, 20, 21 cm, or other suitable lengths. (See Phillips D E: J R Coll Sung Edinb 1994; 39 (5):295). Outside of the body, both the enteric/aspiration and the pharyngeal tube may include separate ports before the tubes merge at the nasal entrance: the enteric/aspiration tube has a port for feeding the patient and/or removing stomach contents and the pharyngeal tube has a port to connect to a suction vacuum to remove fluid from the back of the throat and/or deliver oxygen to the larynx. The ports may differ in design and connectivity mechanism (i.e., clip versus screw) to avoid connection errors.

In some embodiments, a third tube may be included for supplemental oxygen delivery. The third tube may include a small circumference large enough to flow adequate oxygen to the larynx. The third tube may terminate in openings or branched tubes to release the oxygen.

The outer, pharyngeal tube may be connected to a vacuum suction device generating various degrees of negative pressure gradients within the pharyngeal tube and may include longitudinal, sloped perforations (openings) in the tube segment crossing the pharynx allowing pharyngeal secretions to enter the suction tube and to flow for removal into the suction container. Once the combination tube is inserted and connected to the suctioning system secretions aspirated through the perforations of the pharyngeal tube will be suctioned out of the pharynx and through the tube into the suction container. To avoid suction-induced mucosal adherence (sticking) of the pharyngeal tube openings against the mucosa the perforations may run longitudinally and in a sloped (twisted) pattern. In some embodiments, the suction openings may include a “V” shaped pattern. For instance, at the inner surface of the suction tube the opening may be relatively small and extend/expand in diameter (with increasing radius) towards the outer (mucosal) surface of the suction tube. Further, for safety the end of the pharyngeal tube may end in a conically, soft, non-abrasive fashion onto the surface of the feeding tube at the level of the esophagus (about 19, 20, 21, or 22 cm from the nasal entrance).

Device suctioning can be tailed to a patient's needs, such as, for example, suctioning may be configured to be intermittent, constant, variable suctioning strength and durations, and on-demand (i.e. patient or caregiver triggered). Accordingly, there is no need to constantly reinsert a catheter through the nose or mouth to perform suctioning, therefore, avoiding most of the problems with prior devices.

The proposed device has great potential to reduce aspiration events and subsequent aspiration-induced lung injury (e.g., pneumonia, and the like). Indirect evidence supports that conventional intermittent oral suctioning is among the greatest aspiration prevention efforts available and part of many prevention protocols. Also, conveniently, the disclosed device, method, and system can be used at any level of patient consciousness (e.g., ranging from fully awake to comatose), and also can be used in combination with any other oral or nasal device such as endotracheal (breathing) tubes, other enteric tubes, drainage catheters, scopes, and the like. In addition, oral suctioning can always be performed additionally if required.

A presence or absence and recovery state of swallowing reflexes can conveniently be monitored utilizing very small amounts of sterile water injected through the suction port. The small amount of injected water will elicit visible swallowing if the mechanism is recovered. If swallowing is absent or incompetent the injected fluids can readily be re-aspirated through the suction tube port. Also, for the first time, this method will allow to quantify oral secretions (that is amount of fluid pooling within the oropharynx) simply by measuring the secretions amount collected in the suction container. These two measures, reflex testing and secretion monitoring can readily be standardized to employed to monitor swallowing function (and hence determine timely reintroduction of oral feeding). Currently, a standardized method does not exist.

The proposed device may include a simple tube-in-tube design with separate ports; and may be manufactured from materials, such as, for example, commercially available feeding tubes that are currently manufactured from so that they can be readily sterilized.

In some embodiments, the combination tube follows the same insertion procedure currently approved for enteric tube placement. Compared to traditional feeding tubes, the proximal end of the tube may be larger in diameter to accommodate the tube-in-tube design, however; experience shows that such size increases are without clinical consequence as many clinically approved devices with much larger diameters are inserted through the nose: endoscopes, endotracheal tubes, ultrasound transducers, nasal trumpets, and the like.

The disclosed device, method, and system may be designed as add-on device to piggyback on already existing feeding tubes or as a stand-alone device, i.e., for temporary pharyngeal suctioning. The device also increases patient autonomy by allowing the patient to push a button to turn on suctioning. As this method likewise allows hands free, constant suctioning, it will facilitate a secretion-free environment during pharyngeal and laryngeal procedures (e.g., during bronchoscopy, and the like.)

The implementation of these devices and methods will result in measurable outcome improvements such as reducing acquired hospital complications (aspiration events, ventilator-associated pneumonia), patient comfort and autonomy, reduction in caretaker time, reduction in daily suctioning equipment sets, among others. Further, it introduces a more direct, more predictable, and less cumbersome approach to supplement oxygen into the laryngeal entrance.

### BRIEF DESCRIPTION OF THE DRAWINGS

The accompanying drawings, which are incorporated in and constitute a part of this specification, exemplify the embodiments of the present disclosure and, together with the description, serve to explain and illustrate principles of the disclosure. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 illustrates an example of a combination enteric feeding tube and pharyngeal suctioning tube that is constructed in accordance with the principles of the present disclosure;

FIG. 2 illustrates an example of an overview of a device placed into the human body that is constructed in accordance with the principles of the present disclosure; and

FIG. 3 illustrates an example of a cross sectional view of a combination enteric tube and pharyngeal suctioning/oxygenation tube prior to assembly that is constructed in accordance with the principles of the present disclosure.

FIG. 4 illustrates an example of a cross sectional view of a combination enteric tube and pharyngeal suctioning/oxygenation tube prior to assembly with a third oxygen tube that is constructed in accordance with the principles of the present disclosure.

FIG. 5 illustrates an example of a cross sectional view of a combination enteric tube and pharyngeal suctioning/oxygenation tube prior to assembly with an exploded view of a cross section of the pharyngeal tube that is constructed in accordance with the principles of the present disclosure.

FIG. 6 illustrates an example of a cross sectional view of a combination enteric tube, pharyngeal suctioning/oxygenation tube and a third oxygenation tube, with an exploded view of a cross section of the pharyngeal tube that is constructed in accordance with the principles of the present disclosure.

FIG. 7 illustrates an example of a cross sectional view of a combination enteric tube and pharyngeal suctioning/oxygenation tube that is constructed in accordance with the principles of the present disclosure.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein include the same meaning as commonly understood by one of ordinary skill in the art to which this disclosure belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present disclosure. Indeed, the present disclosure is in no way limited to the methods and materials specifically described.

In some embodiments, properties such as dimensions, shapes, relative positions, and so forth, used to describe and claim certain embodiments of the disclosure are to be understood as being modified by the term “about.”

Various examples of the disclosure will now be described. The following description provides specific details for a thorough understanding and enabling description of these examples. One skilled in the relevant art will understand, however, that the disclosure may be practiced without many of these details. Likewise, one skilled in the relevant art will also understand that the disclosure can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the disclosure. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any disclosures or of what may be claimed, but rather as descriptions of features specific to particular implementations of particular disclosures. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Overview

The disclosed systems, devices, and methods provide a simple, safe, effective and widely applicable method-device to suction the throat in patients with absence or incompetent swallowing reflexes. For instance, disclosed is a combination enteric feeding and pharyngeal suctioning tube that both pumps food to a patient's stomach while suctioning a patient's throat to remove pharyngeal secretions. The suctioning tube may be an attachment to a feeding tube that terminates just at or above the esophageal opening. The end of the pharyngeal suctioning tube may include perforations that allow the pharyngeal secretions to be vacuumed into the suctioning portion for removal. The perforations in the pharyngeal tube may run longitudinally and in a sloped (twisted) pattern to avoid suction induced adherence to the mucosal wall of the throat.

The tubes may include coaxially arranged lumens with the pharyngeal suctioning lumen positioned as the outer lumen or external to the feeding tube lumen. The pharyngeal lumen provides a path for secretions that include been vacuumed into the perforations to flow towards an external reservoir for disposal. The pharyngeal tube terminates at its distal end in a smooth, conical fashion, tapering into the feeding tube.

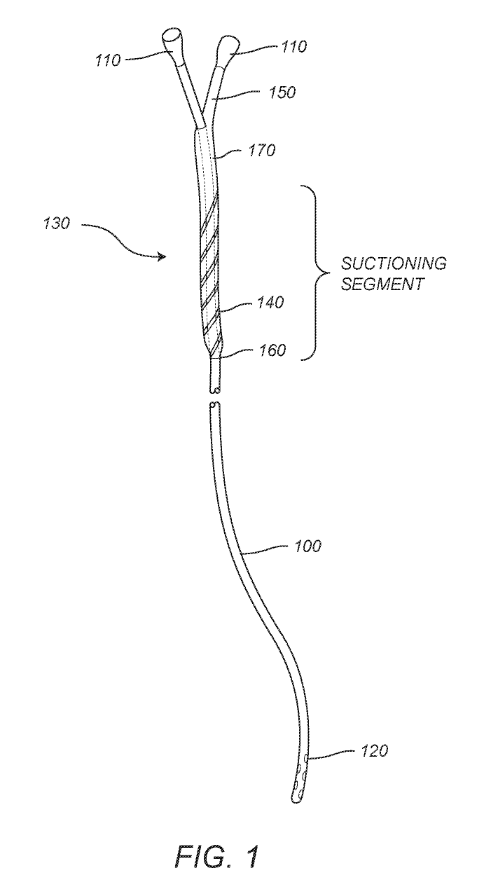

FIG. 1 illustrates an example of the combination feeding/aspiration enteric tube 100 and pharyngeal suction/oxygen tube 150 that are constructures in accordance with the principles of the present disclosure. As illustrated, an enteric feeding/aspiration tube 100 may include a port connector 110 that may be connected to a supply of liquid food that may travel down the enteric tube 100 and exit the tube by holes, openings, or food ports 120 near a distal end of the enteric tube 100 as shown. In some embodiments, the enteric tube 100 may include a standard or conventional feeding tube that is inserted through the nostrils so that the distal end may empty into a patient's stomach. In some embodiments, the enteric tube 100 may be utilized for delivering other substances (e.g., medicine) to the digestive tract of the patient.

In other embodiments, the enteric tube 100 may include an aspiration tube 100 that may suction the contents of the stomach in an emergency or other situations. In this embodiment, size of the lumen of the aspiration tube 100 may be greater than size of the enteric tube and may contain ports 120 that are larger in diameter and/or more numerous than for the feeding tube 100.

### Suction/Oxygen Tube

A suction/oxygen tube 150 is additionally illustrated that may include a suction section 130 (or oxygenation) with perforations 140 that run along the suction section 130. The suction tube 250 may be positioned longitudinally relative to the enteric feeding/aspiration tube 100 so that the suction section 130 is near or at the level of the pharynx and hypopharyns (see diagram) of a patient when the enteric feeding/aspiration tube 100 is inserted into the patient's stomach. In some embodiments, the suction/oxygen tube 150 may be adjustably connected to the enteric tube 100 to allow different suction depths once the suction tube 150 is connected to the enteric tube 100. In other embodiments, the perforations 140 may reach down into the lower end of the esophagus in order to suction regurgitated fluid from the stomach (for example acid reflux related fluid) before it reaches the patient's mouth. In some embodiments, when the suction tube 150 functions as a suctioning device (and not an oxygenation device) it may be positioned with the perforations 140 located in the vicinity of the pharynx. In other embodiments, when the suction/oxygen tube 150 is functioning as an oxygenation tube, it may terminate more closely to the larynx and may therefore be different lengths. In some embodiments, the suction/oxygen tube 150 may be divided into separate components for oxygenation and suctioning which may terminate at different lengths as discussed above and herein. For instance, the oxygen segment may terminate higher than the suctioning segment as secretion will follow gravity and accumulate further down in the throat.

The suction tube 150 may include a conical shaped end 160 that connects to the enteric tube 100 by tapering its diameter to be equivalent to the diameter of the enteric tube 100 at the distal tip of the suction tube 150 as illustrated in FIG. 1. This conical shaped end 160 may help avoid abrasions when inserting the entire tube into a patient's body. Other shapes may be utilized as well that avoid abrasions of the patient, including rounded outward or inward curving, sloping end 160.

The suction tube 150 may also include sealing gaskets or other features to form an airtight seal around the outside of the enteric tube 100 once connected. For example, a hinge mechanism or other features may be utilized to close the suction tube 150 around the enteric tube 100. In other embodiments, claps, clips, elastic parts, or other connectors may be utilized to connect the suction tube 150 with the enteric tube 100.

In some embodiments, the suction tube 150 may move longitudinally with respect to the enteric tube 100 in order to accommodate the relative lengths of a patient's digestive/oral tract while maintaining an airtight seal to ensure sufficient negative pressure develops inside of the suction tube 150 to ingest pharynx secretions. For instance, a gasket or other seal may include a lubricated portion to allow the two to slide with respect to each other. In some embodiments, a caregiver may move the suction tube 150 longitudinally or axially rotate suction tube 150 (or both) to ensure all secretions from the pharynx are appropriately vacuumed.

Once connected, the suction tube 150 may form a lumen 170 around the outside of the enteric tube 100 in a coaxial relationship. This will allow secretions suctioned through the perforations 140 to be transported through the lumen 170 out of the connector port 110 and into a collector. The perforations 140 may include any suitable ports that connect the outside of the suction tube 150 with the lumen 170. In some embodiments, the perforations 140 may be slots or slits that rotate around the suction tube 150 and slope longitudinally in a twisting pattern.

In other embodiments, the suction tube 150 may not include a coaxial relationship and instead may incorporate its own separate lumen 170 that is side-by-side or adjacent to the enteric tube 100. In some embodiments, the suction tube 150 may include a half-moon shaped cross section to allow it to easily connect to the enteric tube 100, while maintaining its own lumen 170. In some embodiments, the suction tube 150 and the enteric tube 100 may be side by side until the suction section 130, where the suction tube 150 could partially or fully surround the enteric tube 100 while maintaining a separate lumen 170 that does not utilize the outside of the enteric tube 100 but has its own lumen 170 defined by its inside edge. Various other combinations could be utilized for connecting the suction tube 150 to the enteric tube 100.

In some embodiments, the suction/oxygenation tube 150 may also deliver oxygen to the larynx either in combination with suctioning (or alternatively) or exclusively. In these embodiments, the perforations 140 could be optimized for the flow of oxygen to deliver the air to the laryngeal entrance.

### System

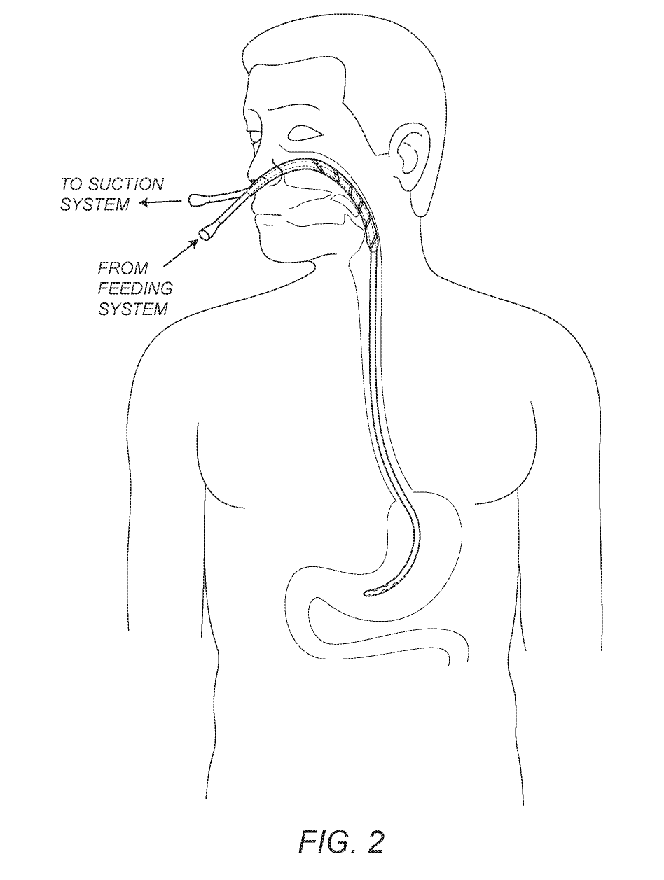

FIG. 2 illustrates an example of a system that is constructed in accordance with the principles of the present disclosure. Illustrated is a suction tube 150 connected to an enteric tube 100 that is inserted into the patient. The suction tube 150 is connected to a vacuum source (not shown) and a collector (not shown) for collecting secretions after they are removed from the patient's pharynx. The enteric tube 100 is connected to a food source and the system can operate as discussed herein and according to convention feeding tubes. Additionally, the suction tube 150 may perform suctioning intermittently, on demand, or by the various other procedures as disclosed herein.

### Assembly

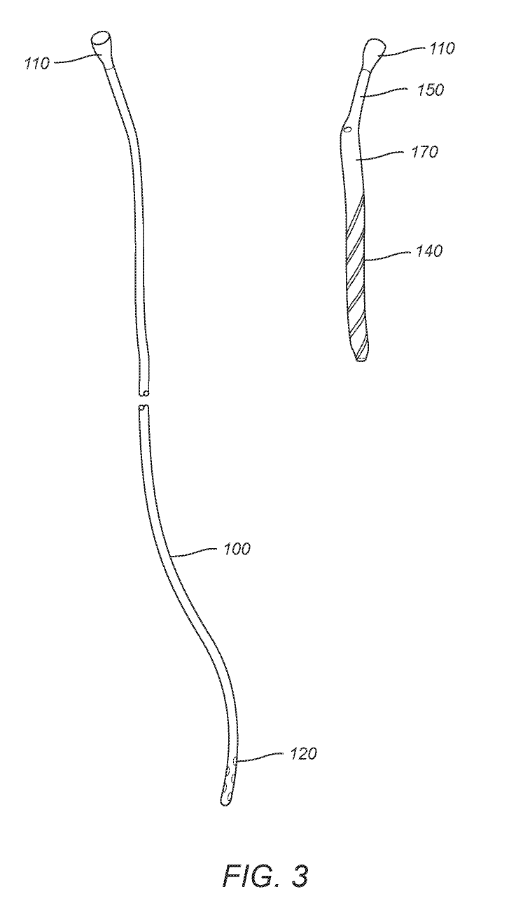

FIG. 3 illustrates an example of a separate suction tube 150 prior to be assembled together with an enteric tube 100 that is constructed in accordance with the present disclosure. In some embodiments, a convention enteric tube 100 may be provided to which a caregiver attaches a suction tube 150. This will allow the disclosed suction tube 150 to be attached to any existing enteric tube 100. In other embodiments, the enteric 100 and suction tubes 150 may be manufactured/assembled together prior to sending to health care facilities.

### Suctioning Methods

A caregiver may connect the suction tube 150 port connector 110 to a vacuum source 210 and a collector 220. Accordingly, when the vacuum source is energized and a vacuum is created inside the suction tube 150 and lumen 170, secretions from a patient's pharynx will be drawn through the perforations 140 through the lumen 170 and to a collector. The caregiver may be able to move the suction tube 150 up and down relative to the enteric tube 100 (which would stay in relatively the same position during this maneuver) to make sure all of the secretions are collected.

The suction tube 150 may include a seal that slidably engages with the enteric tube 100 in order to allow the suction tube 150 to travel up and down relative to the enteric tube 100 without breaking the airtight seal to ensure suction is transferred. In other embodiments, the suction section 130 may rotate around or be rotatable by a caregiver to ensure all of the secretions are vacuumed. In other embodiments, the suction tube 150 and suction section 130 may be fixed along the length of the enteric tube 100 and therefore the entire suction tube 150 and enteric tube 100 complex may be manipulated to ensure the perforations 140 can reach all portions of the pharynx while suctioning is administered.

### Additional Embodiments

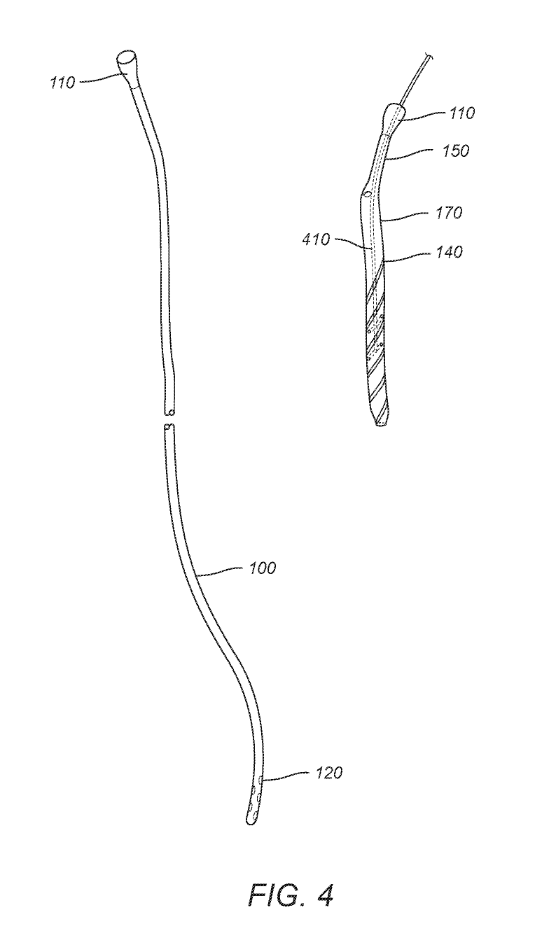

As illustrated in FIG. 4, disclosed herein may include a combination suction tube 150, enteric tube 100, and a third oxygen tube 410 to delivery oxygen to the larynx separately from the suction/oxygenation tube 150. In this embodiment, the oxygen tube 410 may include a relatively small diameter to enable the adequate flow of oxygen to the larynx while avoiding further obstructing the pharynx or other bodily structures as much as possible.

In addition, the oxygen tube 410 may include branched portions at its distal end that may include openings aimed more directly to the larynx. In other embodiments, the oxygen tube 410 may include holes or perforations that are aimed in all directions, to avoid having to circumferentially orienting the oxygen tube 410 with respect to the larynx upon insertion and adjust for different throat lengths. In some embodiments, the oxygen tube 410 portion may end at different lengths than the suctioning tube/oxygenation tube 150. For instance, the oxygen tube 410 may be positioned more towards the larynx while the suctioning tube 150 may be positioned more directly to vacuum secretions of the pharynx.

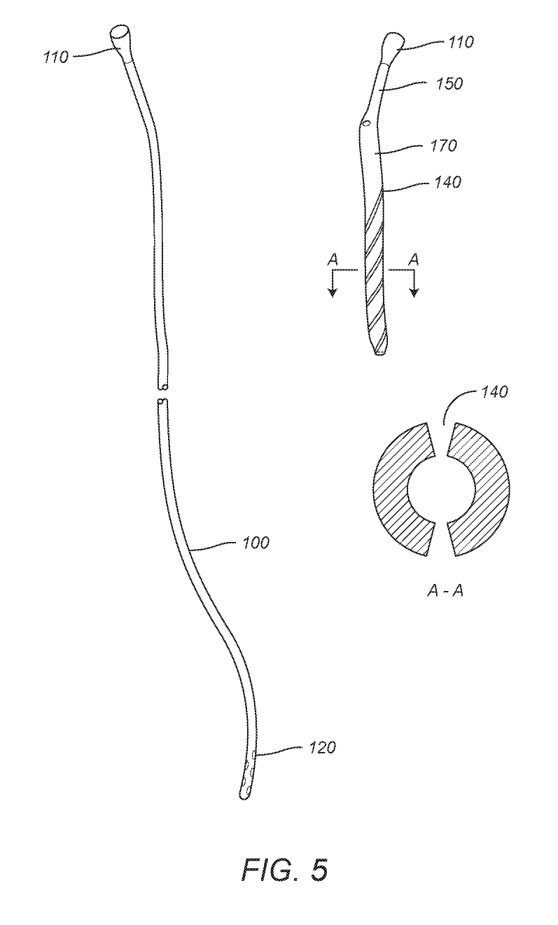

FIG. 5 illustrates another embodiment of the present disclosure that includes a blown-up representation of an axial, cross-sectional view of the perforations 140. As illustrated, the perforations 140 may include a V shaped, a U shaped, or other shaped openings that get wider as the radius increases (or moves away from the center). This decreases the suction pressure by increasing the surface area of suction, which decreases the sticking to the mucosa. Additionally, the narrower inside part of the perforations 140 increases suction pressure at the inner radius of the suction tube 150. In other embodiments, the channels may be straight, or an inverted “V” to get narrower as the radial distance from the center increases.

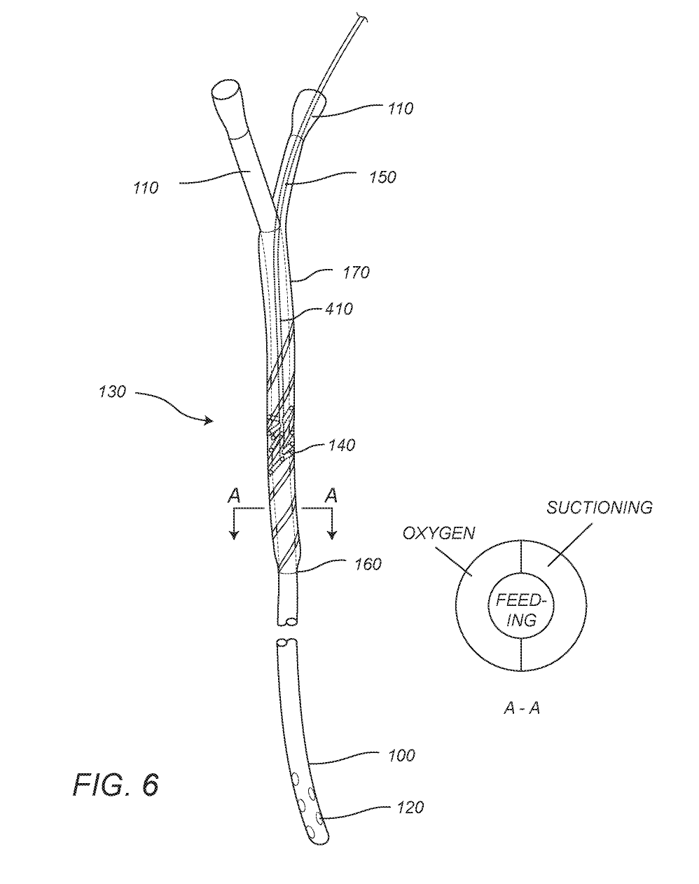

FIG. 6 illustrates an additional embodiment where the enteric tube 100 may be an aspiration tube 100 and/or a feeding tube 100 in combination with the suction tube 150. Additionally, an oxygen tube 410 as disclosed herein may also be added for delivering oxygen to the larynx. This combination device may be useful for addressing emergency situations where aspiration of stomach contents is required, and/or perhaps a patient's blood oxygenation needs to be rapidly increased by delivering high-flow oxygen to the larynx for instance during hypoxemia or even breathing arrest (apnea). For instance, a tube-next-to-tube or tube-in-tube design is disclosed herein that includes the following components (i) a large bore enteric tube 100 to empty the stomach, and (ii) a flow tube 410 ending in a ring-like opening or perforations 140 that delivers high flow, pure oxygen directly to the larynx. In order to optimize the positioning of the oxygen tube to deliver oxygenation to the larynx, one can either track insertion depth and/or monitoring oxygenation response on a device such as a pulse oximeter.

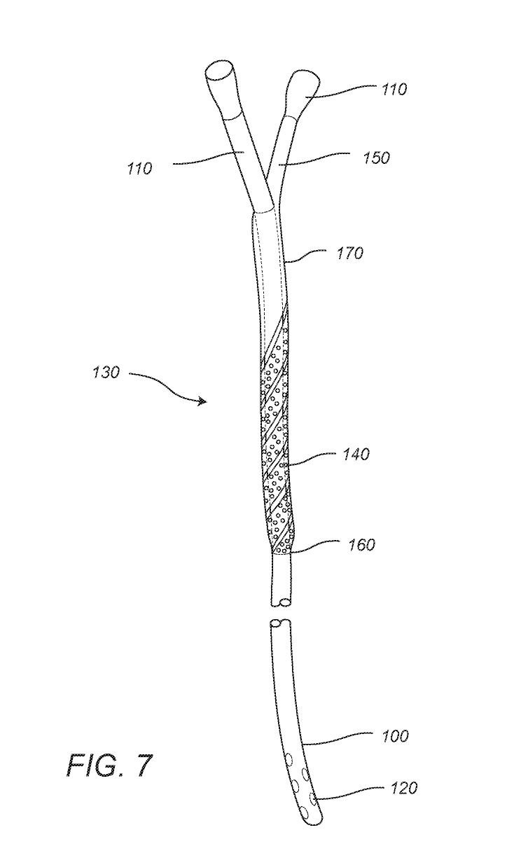

FIG. 7 illustrates an additional embodiment where the enteric tube 100 may be an aspiration tube 100 and/or a feeding tube 100. Additionally, suction/oxygen tube 150 may be modified or optimized for delivering oxygen to the larynx rather than suction. For instance, perforations 140 may be a series (for instance many) holes rather than slots or may be a combination of holes and slots, and may be arranged in an orientation and height that is optimal for delivering oxygen to the larynx.

### Selected Embodiments

Embodiment 1. A suction tube for suctioning a patient's pharynx, the suction tube comprising: a connection port for connecting to a vacuum source and a collector; a suction section; and an enteric tube, wherein the suction section comprises a perforations and a lumen, wherein the perforations is configured to be in fluid communication with the lumen, wherein the lumen is configured to be in fluid communication with the connection port, wherein the suction section is configured to be connectable to outer portion of the enteric tube, wherein when the suction section is configured to be connectable to the enteric tube, and wherein the lumen is retained between the outer portion of the enteric tube and inside of the suction section.

Embodiment 2. The suction tube of embodiment 1, wherein the perforations comprise slots that are formed along the suction section in a longitudinally sloped orientation.

Embodiment 3. The suction tube of embodiment 1, wherein the suction section is slidably connected to the outer portion of the enteric tube to allow the suction tube to slide longitudinally with respect to the enteric tube.

Embodiment 4. The suction tube of embodiment 1, wherein the suction section comprises a tapered section at its distal end that slidably connects to the outer portion of the enteric tube.

Embodiment 5. The suction tube of embodiment 1, wherein the perforations comprise a series of holes.

Embodiment 6. The suction tube of embodiment 1, wherein the perforations comprise a series of slots that are oriented longitudinally along the suction section.

Embodiment 7. The suction tube of embodiment 1, wherein the vacuum source is configured to apply suction to the patient's pharynx via the suction section.

Embodiment 8. The suction tube of embodiment 7, wherein secretions that are collected from the pharynx is deposited into the collector.

Embodiment 9. A combination suction and enteric feeding system for feeding a patient and suctioning the patient's pharynx comprising: an enteric tube comprising an enteric lumen, an opening at a distal end of the enteric tube, a first connection port for connecting the enteric tube to a food source; and a suction tube connected to at least a portion of the enteric tube, wherein the suction tube comprises: a second connection port for connecting the suction tube to a vacuum source; and a suction lumen, wherein the suction lumen is configured to be in fluid communication with the second connection port and perforations that open to outside of the suction tube.

Embodiment 10. The system of embodiment 9, wherein the suction lumen is formed between outside of the enteric tube and inside of the suction tube in a coaxial relationship.

Embodiment 11. The system of embodiment 9, wherein the suction lumen is adjacent to and not coaxial with the enteric lumen.

Embodiment 12. The system of embodiment 9, wherein the suction lumen is formed between at least a portion of outside of the enteric tube and at least a portion of inside of the suction tube in a partially coaxial relationship.

Embodiment 13. The system of embodiment 9, wherein the suction tube includes a hinge and lock mechanism for connecting the suction tube to the enteric tube.

Embodiment 14. The system of embodiment 10, further comprising a gasket which forms an airtight seal on both ends of connected portion of the enteric tube.

Embodiment 15. A method of suctioning a patient's pharynx while feeding the patient comprising: connecting a suction tube to an enteric tube to form a lumen around outer portion of the enteric tube and to form an airtight seal around the enteric tube, wherein the suction tube comprises: perforations that are in fluid communication with the lumen and a connector port that is configured to be in fluid communication with the lumen; inserting the connected suction and enteric tubes into the patient so that a distal end of the enteric tube is positioned in the patient's stomach and the perforations are positioned inside the patient's pharynx; connecting the suction tube to a vacuum source; and initiating suction of the vacuum source to aspire the patient's pharynx and remove secretions in the patient's pharynx.

Embodiment 16. The method of embodiment 15, wherein the enteric tube is configured to be removably connected to a food source so that food can be inserted directly into the patient's stomach through the enteric tube.

Embodiment 17. A method of assembling a suction-enteric tube combination, the method comprising: providing an enteric tube; providing a suction tube having perforations; connecting at least a portion of the suction tube to the enteric tube to form a lumen around the outside of the enteric tube and inside the portion of the suction tube thereby forming an airtight seal at both ends of connected portion of the suction tube.

Embodiment 18. The method of embodiment 17, wherein the perforations are slots formed in a longitudinal, twisting orientation.

Embodiment 19. The method of embodiment 17, wherein the suction tube tapers in diameter at the distal end.

Embodiment 20. The method of embodiment 17, wherein the suction tube may slide longitudinally with respect to the enteric tube while maintaining an airtight seal.

Embodiment 21. A tube for suctioning a patient's pharynx and providing oxygen to the patient's larynx, comprising: a connection port for connecting to a vacuum source, an oxygen source, and a collector; and a flow section with perforations in fluid communication with a lumen, wherein the lumen is in fluid communication with the connection port and a section connectable to outer portion of an enteric tube so that when the flow section is connected to the enteric tube, the lumen is retained between outside of the enteric tube and inside of the suction section.

Embodiment 22. The suction tube of embodiment 21, wherein the perforations comprise slots formed along the suction section in a longitudinally sloped orientation.

Embodiment 23. The suction tube of embodiment 21, wherein the flow section is configured to be slidably connectable to outside portion the enteric tube to allow the tube to slide longitudinally with respect to the enteric tube.

Embodiment 24. The suction tube of embodiment 21, wherein the flow section ends in a tapered section at its distal end.

Embodiment 25. The suction tube of embodiment 21, wherein the perforations comprise a series of holes.

Embodiment 26. The suction tube of embodiment 21, wherein the perforations are a series of slots that are oriented longitudinally along the suction section.

Embodiment 27. A combination suction and enteric system for feeding and aspirating secretions by suctioning a patient's pharynx, as well as providing oxygen to a patient's larynx comprising: an enteric tube comprising an enteric lumen, an opening at a distal end of the enteric tube, and a first connection port for connecting the enteric tube to a food source or a vacuum source; and a flow tube connected to at least a portion of the enteric tube wherein the flow tube comprises: a second connection port for connecting the suction tube to a vacuum source or an oxygen source; and a flow lumen in fluid communication with the second connection port and in fluid communication with perforations that open to the outside of the flow tube.

Embodiment 28. The system of embodiment 27, wherein the flow lumen is formed between an outside of the enteric tube and an inside of the flow tube in a coaxial relationship.

Embodiment 29. The system of embodiment 27, wherein the flow lumen is adjacent to and not coaxial with the enteric lumen.

Embodiment 30. The system of embodiment 27, wherein the flow lumen is formed between at least a portion of the outside of the enteric tube and at least a portion the inside of the flow tube in a partially coaxial relationship.

Embodiment 31. The system of embodiment 27, wherein the flow tube includes a hinge and lock mechanism for connecting the flow tube to the enteric tube.

Embodiment 32. The system of embodiment 27, wherein a gasket forms an airtight seal on both ends of connected portion of the enteric tube.

Embodiment 33. A method of oxygenating a patient's larynx and aspirating the patient's stomach comprising: connecting a flow tube to an enteric tube to form a lumen around an outside of the enteric tube and to form an airtight seal around the enteric tube, wherein the flow tube comprises perforations in fluid communication with the lumen and a connector port in fluid communication with the lumen; inserting the connected flow tube and enteric tube into the patient so that a distal end of the enteric tube is positioned in the patient's stomach and the perforations of the flow tube are positioned inside the patient's pharynx; connecting the flow tube to an oxygen source; and initiating flow of oxygen of the oxygen source to the patient's larynx.

Embodiment 34. The method of embodiment 33, further comprising connecting the enteric tube to a vacuum source and aspiring the patient's stomach.

Embodiment 35. The method of 33, wherein the depth of the flow tube is optimized based on either insertion depth or by monitoring oxygen saturation levels of the patient.

### CONCLUSIONS

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter include been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may include a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application.

Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A suction tube for suctioning a patient's pharynx, the suction tube comprising:
a connection port for connecting to a vacuum source and a collector;
a suction section; and
an enteric tube,
wherein the suction section comprises perforations and a lumen,
wherein the perforations are configured to be in fluid communication with the lumen,
wherein the lumen is configured to be in fluid communication with the connection port,
wherein the suction section is configured to be connectable to outer portion of the enteric tube,
wherein when the suction section is configured to be connectable to the enteric tube, and
wherein the lumen is retained between the outer portion of the enteric tube and inside of the suction section.

2. The suction tube of claim 1, wherein the perforations comprise slots that are formed along the suction section in a longitudinally sloped orientation.

3. The suction tube of claim 1, wherein the suction section is slidably connected to the outer portion of the enteric tube to allow the suction tube to slide longitudinally with respect to the enteric tube.

4. The suction tube of claim 1, wherein the suction section comprises a tapered section at its distal end that slidably connects to the outer portion of the enteric tube.

5. The suction tube of claim 1, wherein the perforations comprise a series of holes.

6. The suction tube of claim 1, wherein the perforations comprise a series of slots that are oriented longitudinally along the suction section.

7. The suction tube of claim 1, wherein the vacuum source is configured to apply suction to the patient's pharynx via the suction section.

8. The suction tube of claim 7, wherein secretions that are collected from the pharynx is deposited into the collector.

9. A combination suction and enteric feeding system for feeding a patient and suctioning the patient's pharynx comprising:
an enteric tube comprising an enteric lumen, an opening at a distal end of the enteric tube, a first connection port for connecting the enteric tube to a food source; and
a suction tube connected to at least a portion of the enteric tube,
wherein the suction tube comprises:
a second connection port for connecting the suction tube to a vacuum source; and
a suction lumen,
wherein the suction lumen is configured to be in fluid communication with the second connection port and perforations that open to outside of the suction tube.

10. The system of claim 9, wherein the suction lumen is formed between outside of the enteric tube and inside of the suction tube in a coaxial relationship.

11. The system of claim 9, wherein the suction lumen is adjacent to and not coaxial with the enteric lumen.

12. The system of claim 9, wherein the suction lumen is formed between at least a portion of outside of the enteric tube and at least a portion of inside of the suction tube in a partially coaxial relationship.

13. The system of claim 9, wherein the suction tube includes a hinge and lock mechanism for connecting the suction tube to the enteric tube.

14. The system of claim 10, further comprising a gasket which forms an airtight seal on both ends of connected portion of the enteric tube.

15. A method of suctioning a patient's pharynx while feeding the patient comprising:
connecting a suction tube to an enteric tube to form a lumen around outer portion of the enteric tube and to form an airtight seal around the enteric tube, wherein the suction tube comprises: perforations that are in fluid communication with the lumen and a connector port that is configured to be in fluid communication with the lumen;
inserting the connected suction and enteric tubes into the patient so that a distal end of the enteric tube is positioned in the patient's stomach and the perforations are positioned inside the patient's pharynx;
connecting the suction tube to a vacuum source; and
initiating suction of the vacuum source to aspire the patient's pharynx and remove secretions in the patient's pharynx.

16. The method of claim 15, wherein the enteric tube is configured to be removably connected to a food source so that food can be inserted directly into the patient's stomach through the enteric tube.

17. A method of assembling a suction-enteric tube combination, the method comprising:
providing an enteric tube;
providing a suction tube having perforations;
connecting at least a portion of the suction tube to the enteric tube to form a lumen around the outside of the enteric tube and inside the portion of the suction tube thereby forming an airtight seal at both ends of connected portion of the suction tube.

18. The method of claim 16, wherein the perforations are slots formed in a longitudinal, twisting orientation.

19. The method of claim 16, wherein the suction tube tapers in diameter at the distal end.

20. The method of claim 17, wherein the suction tube may slide longitudinally with respect to the enteric tube while maintaining an airtight seal.

21. A tube for suctioning a patient's pharynx and providing oxygen to the patient's larynx, comprising:
a connection port for connecting to a vacuum source, an oxygen source, and a collector; and
a flow section with perforations in fluid communication with a lumen,
wherein the lumen is in fluid communication with the connection port and a section connectable to outer portion of an enteric tube so that when the flow section is connected to the enteric tube, the lumen is retained between outside of the enteric tube and inside of the suction section.

22. The suction tube of claim 21, wherein the perforations comprise slots formed along the suction section in a longitudinally sloped orientation.

23. The suction tube of claim 21, wherein the flow section is configured to be slidably connectable to outside portion the enteric tube to allow the tube to slide longitudinally with respect to the enteric tube.

24. The suction tube of claim 21, wherein the flow section ends in a tapered section at its distal end.

25. The suction tube of claim 21, wherein the perforations comprise a series of holes.

26. The suction tube of claim 21, wherein the perforations are a series of slots that are oriented longitudinally along the suction section.

27. A combination suction and enteric system for feeding and aspiring a patient, suctioning a patient's pharynx, and providing oxygen to a patient's larynx comprising:
an enteric tube comprising an enteric lumen, an opening at a distal end of the enteric tube, and a first connection port for connecting the enteric tube to a food source or a vacuum source; and
a flow tube connected to at least a portion of the enteric tube wherein the flow tube comprises:
a second connection port for connecting the suction tube to a vacuum source or an oxygen source; and
a flow lumen in fluid communication with the second connection port and in fluid communication with perforations that open to the outside of the flow tube.

28. The system of claim 27, wherein the flow lumen is formed between an outside of the enteric tube and an inside of the flow tube in a coaxial relationship.

29. The system of claim 27, wherein the flow lumen is adjacent to and not coaxial with the enteric lumen.

30. The system of claim 27, wherein the flow lumen is formed between at least a portion of the outside of the enteric tube and at least a portion the inside of the flow tube in a partially coaxial relationship.

31. The system of claim 27, wherein the flow tube includes a hinge and lock mechanism for connecting the flow tube to the enteric tube.

32. The system of claim 27, wherein a gasket forms an airtight seal on both ends of connected portion of the enteric tube.

33. A method of oxygenating a patient's larynx and aspiring the patient's stomach comprising:
connecting a flow tube to an enteric tube to form a lumen around an outside of the enteric tube and to form an airtight seal around the enteric tube, wherein the flow tube comprises perforations in fluid communication with the lumen and a connector port in fluid communication with the lumen;
inserting the connected flow tube and enteric tube into the patient so that a distal end of the enteric tube is positioned in the patient's stomach and the perforations of the flow tube are positioned inside the patient's pharynx;
connecting the flow tube to an oxygen source; and
initiating flow of oxygen of the oxygen source to the patient's larynx.

34. The method of claim 33, further comprising connecting the enteric tube to a vacuum source and aspiring the patient's stomach.

35. The method of 33, wherein the depth of the flow tube is optimized based on either insertion depth or by monitoring oxygen saturation levels of the patient.
